(19) United States
(12) Patent Application Publication
(10) Pub. No.: US 2009/0209441 A1
(43) Pub. Date: Aug. 20, 2009

(54) Maleated vegetable oils and derivatives, as self-emulsifying lubricants in metalworking

(75) Inventors: Richard M. Lange, Euclid, OH (US); Stuart L. Bartley, Lakewood, OH (US); Christian G. Ollinger, Spartanburg, SC (US); John M. Hogan, Spartanburg, SC (US)

Correspondence Address: THE LUBRIZOL CORPORATION;ATTN: DOCKET CLERK, PATENT DEPT., 29400 LAKELAND BLVD., WICKLIFFE, OH, 44092, US

(73) Assignee: The Lubrizol Corporation, Wickliffe, OH (US)

(21) Appl. No.: 10/585,525

(22) PCT Filed: Jan. 7, 2005
(86) PCT No.: PCT/US05/00487

§ 371 Date: Nov. 29, 2006

Related U.S. Application Data
(60) Provisional application No. 60/535,330, filed on Jan. 9, 2004.

(51) Int. Cl. C10M 129/72; C07C 229/02

(52) U.S. Cl. 508/239; 554/110

(57) ABSTRACT

A succinated triglyceride oil derived from maleating triglyceride oil from a plant or land animal is described for use as an emulsifying agent for metalworking fluids. The metalworking fluid would comprise water; as an emulsifier this succinated triglyceride, optionally further reacted with water, Group IA and IIA metals, ammonium hydroxide, various amines, alkanolamines, alkoxylated alkanolamines, and polyamines to form a modified emulsifier; and optionally an oil and other additives.

### FIELD OF THE INVENTION

The present invention relates to emulsifiers derived from maleating unsaturated triglyceride oils from vegetable sources and land animal sources. The reaction product is a succinated vegetable or animal oil. That reaction product can be further reacted with water, Group IA and IIA metals, ammonium hydroxide, various amines, alkanolamines, and polyamines to form other emulsifiers. The emulsifiers from succinated vegetable or animal oils are particularly useful in water based metalworking fluids.

### BACKGROUND OF THE INVENTION

Mineral oils and vegetable oils are often used as components of formulated aqueous metalworking compositions. In these instances, a discreet emulsifying agent must be used to hold these non-polar, water insoluble oils in uniform suspension in the water matrix, and this adds significant cost to the commercial metalworking formulations. Additionally, not all types of emulsifying agents are equally effective in forming stable dispersions (emulsions) of vegetable oils in water, so it becomes critical to use the correct and most effective surfactant to accomplish this.

The patent literature contains a number of references to maleation of vegetable oils over the past 30 years, some of which may be pertinent, and this is currently being reviewed. Some early patents refer to thermal maleation, some describe the need to use polyunsaturated vegetable oils, some require catalysts to isomerize the two C═C bonds in linoleate moieties into conjugation to allow the Diels-Alder reaction with malan (maleic anhydride) to proceed, and some cite the use of radical initiators, and refer to the vegetable oils as “monomers”, which is (in our opinion) inconsistent with chemical nomenclature.

Most of the applications described in the patent literature involve the use of the maleated vegetable oils in modifying the properties of air-cured alkyd coatings or to give flexibility and improved adhesion to substrates. Other applications include use in inks and coatings as aiding pigment dispersancy, and quite a few address their use in cosmetics and personal care products (emollients and skin softening). One Japanese patent (JP Pub. 4-73477) refers to maleated fish oil in a metalworking fluid.

It would be desirable to have functionalized natural oils for use in metalworking that are self-emulsifying.

### SUMMARY OF THE INVENTION

The reaction product of maleating a triglyceride oil from a plant or land animal was unexpectedly found to be particularly useful emulsifier for oil in water emulsions. The reaction typically results in primarily mono-maleation under mole ratios of 1:1 between reactants, but with some percentage of di-maleation and possibly tri- or higher maleation being possible in still smaller amounts. The triglyceride oils are readily available in a variety of purities and readily undergo thermal maleation. The maleated products are often called succinated triglyceride oils because the maleic anhydride loses one carbon-to-carbon double bond in the thermal coupling reaction, while introducing it (i.e. converting the maleic acid or anhydride to a succinic acid or succinic anhydride substituent). In the present application, the terms “succination” and “maleation” can be used interchangeably to convey the concept and description of the natural oil functionalization.

We have found that these succinated triglycerides may be further reacted with water, Group IA and IIA metals, ammonium hydroxide, various amines, alkanolamines, and polyamines to form new emulsifiers that have different properties due to the addition of larger polar groups from the additional reactant.

Additionally, these functionalized triglyceride (natural) oils can self-emulsify, that is, form an instantaneous emulsion when added to water or other aqueous-based formulation. The nature and ease of formation of the emulsions is dependent on both the extent (degree of) polar functionalization of the triglyceride oils and on the particular base oils used as the dispersed hydrophobic phase.

A preferred application for the succinated triglyceride oils are as emulsifiers in aqueous based metal working fluids. Historically alkyl benzene petroleum sulfonates have been used extensively in metalworking fluids due to their reasonable price, insensitivity to water hardness, good emulsifying capabilities, low tendency to foam, etc. Various oils including vegetable oils were added to the metalworking fluids to provide lubricity during metalworking operations. It has been found that the succinated triglyceride oils are particularly effective in emulsifying vegetable oil-containing metalworking fluids. The combination of vegetable or animal triglycerides and a functionalized vegetable or land animal triglyceride oil offers low foaming tendencies, good lubricity, low toxicity, and constitutes a novel and efficient use for these polar-functionalized triglyceride oils.

### DETAILED DESCRIPTION OF THE INVENTION

The reaction product of maleic anhydride and a triglyceride oil can be made by the thermal condensation of maleic anhydride or other unsaturated carboxylic acid capable of undergoing either “Ene” or “Diels-Alder” adduction to the unsaturated sites in vegetable oils. The modified triglyceride oils become easily self-emulsifiable in water when treated with bases, and do not require any additional emulsifiers. Suitable bases to react with the reaction product of maleic anhydride and a triglyceride oil of a plant or land animal include the Group IA and IIA metals, ammonium hydroxide, various amines, alkanolamines, and polyamines. A preferred triglyceride oil is a triglyceride oil of a plant or of a land animal.

The functionalized triglyceride oils (and their derivatives) described in this process comprise a class of self-emulsifiable lubricants which, depending on their compositions, are also capable of acting as emulsifiers for other additives in the metalworking formulations. They are physically benign agents that help to stabilize emulsions, can add lubricity to the aqueous emulsion formulations, and help to reduce corrosion, while facilitating machining and other metalworking operations. The products should also have a fairly high susceptibility to bio-degradation, particularly in emulsified media and thus are more environmentally friendly (more benign) than some other emulsifiers previously used in metalworking fluids.

The chemistry of manufacture of these self-emulsifying vegetable oils is relatively simple and straightforward. Attachment of maleic anhydride (preferred agent) may be carried out by thermal condensation at temperatures above 100° C. and preferably about 175-250° C., at the unsaturated C═C sites of the fatty acid moieties in the triglyceride oil.

The degree of maleic anhydride coupling to the triglyceride oil is a function of the oil composition and molar charge ratio of maleic anhydride to triglyceride oil. Oils comprising oleic acid esters can condense with maleic anhydride through an “Ene” mechanism (“Alder Reaction”), which involves the same type of electron transfer and subsequent 1,4-hydrogen migration common to the reaction of maleic anhydride with olefins and polyolefins, to form alkenylsuccinic anhydrides.

Triglyceride oils comprising high levels of linoleic acid esters can condense with maleic anhydride in a Diels-Alder manner, to form cyclohexene-dicarboxylic anhydride moieties. During thermal condensation, the two non-conjugated C═C bonds in the linoleic acid esters move into conjugation to form a 1,3-dienoic system, which easily undergoes 1,4-addition with the maleic anhydride C═C bond, to form a 4-cyclohexene-1,2-dicarboxylic anhydride near the midpoint of the linoleate ester “tail”.

With the exception of coconut oil, which is mainly composed of saturated C12-C18 triglycerides, most domestic triglyceride oils from plants (oils from plants are commonly referred to as vegetable oils, a preferred starting material for this disclosure), contain various levels of both the unsaturated oleic and linoleic components. Coconut-derived palm oil and palm kernel oil both contain relatively high levels of saturated C12-14 triglycerides. They also have moderate levels (ca. 17-25%) of reactive oleic and linoleic esters, which can react with maleic anhydride.

High erucic rapeseed oil contains about 50% oleic, linoleic, and linolenic esters, and about 45% mono-unsaturated C22 acid ester, all of which can be thermally maleated.

### Method and Process:

Step A: Triglyceride oils such as from vegetable sources and land animal sources [component 1] containing at least one reactive C═C double bond per molecule are charged to a suitable reaction vessel.

Step B: Maleic anhydride or other unsaturated functional reagent [component 2] capable of acting as a dienophile, or of undergoing “Ene” condensation, is charged to the vessel, and the mixture is stirred and heated to 175-250 C. The mixture is held for 1-5 hours at final temperature to effect the condensations

An inert atmosphere of nitrogen, CO2, or other non-reactive gas can be used during the condensation, to promote the formation of lighter-colored products.

Additional stabilizers and antioxidants may also be optionally present to assure light colored products, and to prevent radical coupling of unsaturated moieties in the reaction mixture.

A small amount (ca. 0.1-5.0%) of a compatibilizing solvent [component 3] may be added, if desired, to promote contact between the polar maleic anhydride and the relatively non-polar olefinic “tails” of the triglyceride, and to inhibit loss of maleic anhydride by sublimation or entrainment at higher temperatures. Suitable solvents include toluene and certain low alkyl esters. During heating, the majority of solvent may be gradually removed from reaction.

Step C: The reaction mixture is sparged or vacuum-stripped after completion to remove solvent and unreacted maleic anhydride.

Step D: Optionally, the stripped product may be cooled somewhat, and filtered for clarity through a simple cartridge filter, usually without the use of filter aid.

The maleated intermediate [component 4] is an anhydride (or carboxylic acid)-grafted vegetable oil which may be used directly as a self-emulsifiable lubricity agent in aqueous emulsions, or which can be converted to esters, partial esters, amidic acids, amidic esters, imides, and other derivatives that are also useful in metalworking compositions, as lubricants, lubricity agents, friction modifiers, and antiwear agents.

### Reagents Used:

Component 1. Triglyceride Oils

Includes triglycerides such as soybean oil (“SYBO”), rapeseed oil, canola oil, safflower oil, corn oil, chicken fat, butter oil, cottonseed oil, sunflower oil, high oleic sunflower oil, peanut oil, palm oil, palm kernel oil, olive oil, and other natural oils derived from plants and land animals which contain measurable amounts of C═C unsaturation. Fully saturated triglycerides such as coconut oil are not included within the scope of these thermal functionalizations. Although fish oils may have more reactive unsaturation than vegetable and plant oils, they also, have higher odor and tendency to degrade, making them less desirable. Desirably at least 20 mole percent of the fatty acids of the triglyceride oil is unsaturated and more desirably at least 50 moles percent.

Component 2. Unsaturated Acid or Anhydride Functionalizing Agent

Maleic anhydride is the preferred component 2, but other unsaturated acids or anhydrides are also useful, including but not limited to, at least one of maleic acid, fumaric acid, itaconic acid and anhydride, acrylic acid, cinnamic acid, and crotonic acid.

The amount of Component 2 used can be based on the degree and type of unsaturation in the vegetable oil, and the degree of functionalization (i.e., total acid number in mgKOH/g, TAN) desired for the immediate purpose. On a molar basis, this would amount to a practical amount of from about 0.02-to-2.5 mole of maleic anhydride (more broadly unsaturated acid or anhydride functionalizing agent) per mole of vegetable or land animal (natural) oil.

Component 3. The Optional Compatibilizing Solvent

Suitable solvents are those which can dissolve maleic anhydride or other suitable unsaturated carboxylic acid, anhydride, ester, or amide, without reacting with either the grafting agent or the vegetable oil substrate. Preferably they are volatile so they can be easily removed either during, or after reaction is complete. Examples of solvents include cyclohexane, toluene and similar lower alkyl aromatics, ketones and low alkyl esters.

The amount of Component 3 useful in these processes is generally low, on the order of about 0.1% to about 5%, based on the amount of natural oil.

### Derivatives of the Functionalized Natural Oils

Carboxylic Acids: The maleated intermediates (component 4) may be reacted with water, generally in the amount of about 1 mole per anhydride present. Mono-maleated vegetable oils will yield dicarboxylic acids, di-maleated vegetable oils will yield tetra-carboxylic acids, etc.

Esters: Reaction of maleated intermediates with alcohols produces esters. Depending on the amount of ester linkages desired and the desire or lack thereof the amount of alcohols can vary from a molar ratio of alcohol to succinate groups on the maleated intermediate from about 0.1 (only about 10% of the succinate groups would partially esterified) to about 2 (where near about 100% of the succinate groups would be esterified). The presence of polyfunctional alcohols, amines, or alkanol amines further complicates things as polyfunctional alcohols (or amines or some alkanolamines) can serve as coupling agents for the maleated intermediate. Alkanolamines may also partially salt (either internally (alcohol of alkanolamine forms ester linkage for one carboxylic acid of succinic anhydride or acid as amine of alkanolamine forms salt with the remaining carboxylic acid on the same succinic molecule) or externally (alcohol portion of alkanolamine not otherwise associated with succinic acid group which is salted by the amine of the alkanolamine)). These alcohols can include (but are not limited to) alkanolamines; amine ethoxylates; monohydridic alcohols, diols, and polyols; condensation products of polyols; and poly(alkylene oxide) and its derivatives. Reaction of one mole of alcohol per anhydride gives half-esters/half acids, which can be easily emulsified in water, using bases. Reaction with greater amounts of alcohols can produce mixtures of diesters and half-esters within the same molecule. Alkoxylated amines can react with the anhydride groups to produce aminoalkyl esters, which may be emulsified or dispersed in water. Alkoxylated amines may be reacted with ethylene oxide or propylene oxide to form ethoxylates that can be coupled to the succinated vegetable oils. These ethoxylated or propoxylated amines can dramatically change the water solubility of the succinated vegetable oils.

Amidic Acids and Imides: Reaction of the anhydride portion of the maleated intermediates with secondary amines produces N,N-dialkyl amidic acids, which can be easily emulsified in water by treatment with bases. Reaction of the anhydride with primary amines will initially produce N-alkyl amidic acids, which may close down to produce imide functions. Depending on the nature of the primary amine used, the vegetable oil imides may, or may not be water-dispersible.

Polyetheramines such as the commercial “Jeffamines” from Huntsman, or those from Tomah, can produce water-dispersible amidic acid and imide derivatives.

The maleated vegetable oils and their derivatives can be easily self-emulsified in water by treatment with bases, and are useful at levels of about 0.5 or 1 to about 10 weight percent, more desirably from about 2 to about 8 weight percent as lubricity agents and additives for metalworking.

See TABLES 1 and 2 below.

TABLE 1
Properties of Various Functionalized Natural Oils

| Oil/Maleic | | | | |
|---|---|---|---|---|
| Anhydride/ | | | | |
| Amine or | | High Oleic | | |
| Hydroxide | Rapeseed Oil, | Sunflower Oil, | Soybean Oil, | Soybean Oil, |
| Mole Ratio | 1:1:2 | 1:1:2 | 1:1:2 | 1:2:4 |

TEA Ester/Salt

| | | | | |
|---|---|---|---|---|
| pH @ 5% in tap | 8.3 | 8.4 | 8.3 | 8.6 |
| water | | | | |
| Emulsion | Milky white | Milky white | Milky/semi- | Semi- |
| Appearance (5% | | | translucent | translucent |
| in tap water) | | | | |

Emulsion Stability (mL oil/total mL of separated materials) based on IP 263

| | | | | |
|---|---|---|---|---|
| 5% in tap water, | 0/0.2 | 0/0.1 | 0/0.1 | 0/0 |
| 22° C. | | | | |
| 5% in 400 ppm | 0.2/0.9 | 0/5 | 0/0.4 | 0/0 |
| water (in terms | | | | |
| of CaCO₃), 40 | | | | |
| ° C. | | | | |

Emulsification Power (mL oil/total mL of separated materials)

| | | | | |
|---|---|---|---|---|
| @ 20% in | 0/>5 | 0/>5 | Not Soluble | Not Soluble |
| naphthenic oil, | | | | |
| 5% in tap water | | | | |
| @ 20% in | Not Soluble | Not Soluble | Not Soluble | Not Soluble |
| paraffinic oil, 5% | | | | |
| in tap water | | | | |
| @ 20% in high | Not Soluble | Not Soluble | Not Soluble | Not Soluble |
| oleic canola oil, | | | | |
| 5% in tap water | | | | |

Iron Chip Corrosion Test (% area of staining) based on IP 287

| | | | | |
|---|---|---|---|---|
| 3% in 200 ppm | 30% | 20% | 20% | 20% |
| water (in terms | | | | |
| of CaCO₃) | | | | |
| 5% in 200 ppm | <1% | <1% | <1% | <1% |
| water (in terms | | | | |
| of CaCO₃) | | | | |

IP 312 Foam Test

| | | | | |
|---|---|---|---|---|
| V_{0 min}, V_{5 min}, | 20, 10, 8, 6 | 10, 6, —, — | 50, 4, 4, 4 | 96, 84, 62, 40 |
| V_{10 min}, V_{15 min} | | | | |
| Time for foam to | — | 8 min, 10 s | — | — |
| collapse | | | | |

MEA Ester/Amide/Salt

| | | | | |
|---|---|---|---|---|
| pH @ 5% in tap | 9.0 | 9.2 | 9.2 | 9.3 |
| water | | | | |
| Emulsion | Milky amber | Semi- | Semi- | Translucent |
| Appearance (5% | | translucent | translucent | |
| in tap water) | | | | |

Emulsion Stability (mL oil/total mL of separated materials) based on IP 263

| | | | | |
|---|---|---|---|---|
| 5% in tap water, | 0/0 | 0/0 | 0/0 | 0/0 |
| 22° C. | | | | |
| 5% in 400 ppm | 0/0 | 0/0 | 0/0 | 0/0 |
| water (in terms | | | | |
| of CaCO₃), 40 | | | | |
| ° C. | | | | |

Emulsification Power (ml oil/total mL of separated materials)

| | | | | |
|---|---|---|---|---|
| @ 20% in | Does Not | 0/5 | 0/>5 | 0/>5 |
| naphthenic oil, | Emulsify | | | |
| 5% in tap water | | | | |
| @ 20% in | Not Soluble | 0/5.2 | 0/>5 | 0/>5 |
| paraffinic oil, 5% | | | | |
| in tap water | | | | |
| @ 20% in high | Not Soluble | Not Soluble | Not Soluble | Not Soluble |
| oleic canola oil, | | | | |
| 5% in tap water | | | | |

Iron Chip Corrosion Test (% area of staining) based on IP 287

| | | | | |
|---|---|---|---|---|
| 3% in 200 ppm | 10% | 10% | 10% | 20% |
| water (in terms | | | | |
| of CaCO₃) | | | | |
| 5% in 200 ppm | 3% | 5% | 0% | 10% |
| water (in terms | | | | |
| of CaCO₃) | | | | |

IP 312 Foam Test

| | | | | |
|---|---|---|---|---|
| V_{0 min}, V_{5 min}, | 150, 80, 40, 30 | 90, 54, 38, 34 | 150, 68, 42, 32 | 142, 126, 78, 48 |
| V_{10 min}, V_{15 min} | | | | |
| Time for foam to | — | — | — | — |
| collapse | | | | |

KOH Salt

| | | | | |
|---|---|---|---|---|
| pH @ 5% in tap | 10.3 | 10.4 | 11.0 | 9.0 |
| water: | | | | |
| Emulsion | Milky | Milky/semi- | Semi- | Semi- |
| Appearance (5% | | translucent | translucent | translucent |
| in tap water) | | | | |

Emulsion Stability (mL oil/total mL of separated materials) based on IP 263

| | | | | |
|---|---|---|---|---|
| 5% in tap water, | 0/0.5 | 0/0 | 0/0 | 0/0 |
| 22° C. | | | | |
| 5% in 400 ppm | 0/0 | 0/0 | 0/0 | 0/0 |
| water (in terms | | | | |
| of CaCO₃), 40 | | | | |
| ° C. | | | | |

Emulsification Power (mL oil/total mL of separated materials)

| | | | | |
|---|---|---|---|---|
| @ 20% in | Not Soluble | Not Soluble | Not Soluble | Not Soluble |
| naphthenic oil, | | | | |
| 5% in tap water | | | | |
| @ 20% in | Not Soluble | Not Soluble | Not Soluble | Not Soluble |
| paraffinic oil, 5% | | | | |
| in tap water | | | | |
| @ 20% in high | Not Soluble | Not Soluble | Not Soluble | Not Soluble |
| oleic canola oil, | | | | |
| 5% in tap water | | | | |

Iron Chip Corrosion Test (% area of staining) based on IP 287:

| | | | | |
|---|---|---|---|---|
| 3% in 200 ppm | 5% | 20% | 15% | 15% |
| water (in terms | | | | |
| of CaCO₃) | | | | |
| 5% in 200 ppm | 0% | 10% | 10% | 5% |
| water (in terms | | | | |
| of CaCO₃) | | | | |

IP 312 Foam Test:

| | | | | |
|---|---|---|---|---|
| V_{0 min}, V_{5 min}, | >150, 130, | >150, 150, | >150, 150, | >150, >150, |
| V_{10 min}, V_{15 min} | 60, 30− | 94, 70 | 80, 70 | >150, >150 |
| Time for foam to | — | — | — | — |
| collapse | | | | |

3 Mole EO TEA Ester/Salt

| | | | | |
|---|---|---|---|---|
| pH @ 5% in tap | 8.0 | 8.0 | 8.0 | 8.0 |
| water: | | | | |
| Emulsion | Semi- | Semi- | Semi- | Semi- |
| Appearance (5% | translucent | translucent | translucent | translucent |
| in tap water) | | | | |

Emulsion Stability (mL oil/total mL of separated materials) based on IP 263

| | | | | |
|---|---|---|---|---|
| 5% in tap water, | 0/0 | 0/0 | 0/0 | 0/0 |
| 22° C. | | | | |
| 5% in 400 ppm | 0/0 | 0/0.2 | 0/0 | 0/0 |
| water (in terms | | | | |
| of CaCO₃), 40 | | | | |
| ° C. | | | | |

Emulsification Power (mL oil/total mL of separated materials)

| | | | | |
|---|---|---|---|---|
| @ 20% in | Not Soluble | 0/>5 | Not Soluble | Not Soluble |
| naphthenic oil, | | | | |
| 5% in tap water | | | | |
| @ 20% in | Not Soluble | Not Soluble | Not Soluble | Not Soluble |
| paraffinic oil, 5% | | | | |
| in tap water | | | | |
| @ 20% in high | Not Soluble | Not Soluble | Not Soluble | Not Soluble |
| oleic canola oil, | | | | |
| 5% in tap water | | | | |

Iron Chip Corrosion Test (% area of staining) based on IP 287

| | | | | |
|---|---|---|---|---|
| 3% in 200 ppm | 90% | 80% | 95% | 80% |
| water (in terms | | | | |
| of CaCO₃) | | | | |
| 5% in 200 ppm | 70% | 40% | 90% | 70% |
| water (in terms | | | | |
| of CaCO₃) | | | | |

IP 312 Foam Test:

| | | | | |
|---|---|---|---|---|
| V_{0 min}, V_{5 min}, | 38, 28, 26, 24 | 50, 16, 12, 10 | 34, —, —, — | 64, 46, 42, 40 |
| V_{10 min}, V_{15 min} | | | | |
| Time for foam to | — | — | 22 s | — |
| collapse | | | | |

TABLE 2
Properties of Various Functionalized Natural Oils (Cont.)

| Oil/Maleic | | | |
|---|---|---|---|
| Anhydride/ | | | |
| Amine or | | | |
| Hydroxide Mole | Sunflower Oil, | Sunflower Oil, | |
| Ratio | 1:1:2 | 1:1.5:3 | Lard Oil, 1:1:2 |

TEA Ester/Salt

| | | | |
|---|---|---|---|
| pH @ 5% in tap | 8.5 | 8.4 | 8.5 |
| water | | | |
| Emulsion | Milky, Semi- | Translucent | Milky, amber |
| Appearance (5% | translucent | | |
| in tap water) | | | |

Emulsion Stability (mL oil/total mL of
separated materials)based on IP 263

| | | | |
|---|---|---|---|
| 5% in tap water, | 0/0 | 0/0 | 0/0 |
| 22° C. | | | |
| 5% in 400 ppm | 0/0.1 | 0/0 | 4.8/4.8 |
| water (in terms | | | |
| of CaCO₃), 40 | | | |
| ° C. | | | |

Emulsification Power (mL oil/total mL of separated materials)

| | | | |
|---|---|---|---|
| @ 20% in | Not Soluble | Not Soluble | 0/0.2 |
| naphthenic oil, | | | |
| 5% in tap water | | | |
| @ 20% in | Not Soluble | Not Soluble | Not Soluble |
| paraffinic oil, 5% | | | |
| in tap water | | | |
| @ 20% in high | Not Soluble | Not Soluble | Not Soluble |
| oleic canola oil, | | | |
| 5% in tap water | | | |

Iron Chip Corrosion Test (% area of staining) based on IP 287

| | | | |
|---|---|---|---|
| 3% in 200 ppm | 20% | 40% | 10% |
| water (in terms | | | |
| of CaCO₃) | | | |
| 5% in 200 ppm | <1% | 3% | 2% |
| water (in terms | | | |
| of CaCO₃) | | | |

IP 312 Foam Test

| | | | |
|---|---|---|---|
| V_{0 min}, V_{5 min}, | 30, 10, 6, 4 | 80, 28, 14, 10 | 10, 0, 0, 0 |
| V_{10 min}, V_{15 min} | | | |
| Time for foam to | — | — | 30 s |
| collapse | | | |

### EXAMPLES

### Example 1

### Soybean Oil/Maleic Anhydride/Triethanolamine (1:1:2) Mole

With stirring and under a nitrogen atmosphere, maleic anhydride is reacted with soybean oil on a one-to-one mole basis at 220° C. for four hours to form the maleated soybean oil. A small amount of toluene (0.25% wt) is added prior to heating so that the maleic anhydride will not sublime and be lost. When cooled, this intermediate is further reacted with one mole of triethanolamine for every mole of maleated soybean oil at 50° C. for one hour to form the ester. The resulting functionalized soybean oil self-emulsifies well in water when mixed at 5% in water and has an approximate pH of 8.4. Emulsion testing using a modified IP 263 method (at 5% treat level) showed no oil or cream formation in synthetic 400 ppm water (in terms of CaCO₃) at 40° C. or in 30 ppm hardness water at 22° C. after 24 hours. Due to the high viscosity of these functionalized natural oils, the emulsion stabilities for all examples were tested using a modified IP 263 method. The standard test method requires two minutes of mixing after the last drop of fluid is added to water. For these examples, mixing was continued until homogeneous. Foaming tests (by method IP 312, IP stands for The Institute of Petroleum, the UK's version of standardized tests for the petroleum industry) on the 5% in water emulsion showed that foam collapse was quite significant. Corrosion properties were measured using the IP 287 method and the result for this example was a break-point of 4%. For all other examples, the percentage area of staining was recorded at 3% and 5% dilutions to determine relative corrosion properties.

### Example 2

### Rapeseed Oil/Maleic Anhydride/Mono-Ethanolamine (1:1:2) Mole

With stirring and under a nitrogen atmosphere, Maleic anhydride is reacted with rapeseed oil on a one-to-one mole basis at 220° C. for four hours to form the maleated rapeseed oil. A small amount of toluene (0.25% wt) is added prior to heating so that the maleic anhydride will not sublime and be lost. When cooled, this intermediate is further reacted with two moles of monoethanolamine at 40° C. (exotherm occurs so the addition of monoethanolamine is added over a one-hour period) and heated ˜50° C. for an additional hour after all the monoethanolamine is added to form the partial ester-amide-salt. The resulting functionalized rapeseed oil self-emulsifies well in water when mixed at 5% in water and has an approximate pH of 10.0. Emulsion testing using the modified IP 263 method mentioned in Example 1 (at 5% treat level) showed no oil and approximately 1.4 mL of cream formation in synthetic 400 ppm CaCO₃ water at 40° C. after 24 hours. This functionalized rapeseed oil demonstrated that it can also serve as an emulsifier for paraffinic and naphthenic oils in an aqueous medium.

The anti-misting polymer of U.S. Pat. No. 6,100,225, hereby incorporated by reference for its teaching on its anti-mist agent, is a useful additive in this formulation. It or other polymeric anti-mist agents can be used in a concentration range of 0.02 weight percent to 10 weight percent based upon the total weight of the composition.

In addition to the anti-misting polymer, the aqueous metal working fluids may contain additives to improve the properties of the composition. These additives include anti-foam agents, metal deactivators, and corrosion inhibitors, antimicrobial, anticorrosion, extreme pressure, antiwear, antifriction, and antirust agents. Such materials and the relative amounts used in metalworking fluids are well known to those skilled in the art.

The metal working fluids of the present invention may also be oil-in-water emulsions. The emulsion compositions metalworking fluids contain the same types and amounts of optional additives as the purely aqueous compositions discussed above. The particular maleated triglyceride oils of this application show enhancements over other purely aqueous metalworking compositions due to the inherent lubricity of the maleated triglyceride oils relative to other emulsifiers for these fluids. The compositions may also contain the property improving additives which have been used in the purely aqueous fluids.

The oils used in the emulsion compositions may include vegetable oils as previously defined including triglyceride oils from animals, petroleum oils, such as oils of lubricating viscosity, crude oils, diesel oils, mineral seal oils, kerosenes, fuel oils, white oils, naphthenic oils, and aromatic oils. Liquid oils include natural lubricating oils, such as (land) animal oils, vegetable oils, mineral lubricating oils, solvent or acid treated mineral oils, oils derived from coal or shale, and synthetic oils. Synthetic oils include hydrocarbon oils and halo-substituted hydrocarbon oils such as polymerized and interpolymerized olefins, for example polybutylenes, polypropylenes, propylene-isobutylene copolymers, chlorinated polybutylenes, poly(1-hexenes), poly(1-octenes), poly(1-decenes); alkyl benzenes, such as dodecylbenzenes, tetradecylbenzenes, dinonylbenzenes, di-(2-ethylhexyl)benzenes; polyphenyls such as biphenyls, terphenyls, and alkylated polyphenyls; and alkylated diphenyl ethers and alkylated diphenyl sulfides and derivatives, analogs and homologs thereof. Vegetable oils and triglyceride oils from animals are preferred in some applications due to their biodegradability and benign effect on most water treatment processes and ecosystems.

Alkylene oxide polymers and derivatives thereof where terminal hydroxy groups have been modified by esterification, etherification etc. constitute another class of synthetic oils. These are exemplified by polyoxyalkylene polymers prepared by the polymerization of ethylene oxide or propylene oxide, the alkyl and aryl ethers of these polyoxyalkylene polymers such as methyl-polyisopropylene glycol ethers, diphenyl and diethyl ethers of polyethylene glycol; and mono and polycarboxylic esters thereof, for example, the acetic esters, mixed C₃-C₈, fatty acid esters and C₁₃ OxO diester of tetraethylene glycol. Simple aliphatic ethers may be used as synthetic oils, such as, dioctyl ether, didecyl ether, di(2-ethylhexyl)ether.

Another suitable class of synthetic oils comprises the esters of fatty acids such as ethyl oleate, lauryl hexanoate, and decyl palmitate. The esters of dicarboxylic acids such as phthalic acid, succinic acid, maleic acid, azelaic acid, sebacic acid, fumaric acid, adipic acid, linoleic acid dimer, malonic acid, alkyl malonic acids, alkenyl malonic acids with a variety of alcohols such as butyl alcohol, hexyl alcohol, dodecyl alcohol, 2-ethylhexyl alcohol, ethylene glycol, diethylene glycol monoethyl ether, propylene glycol. Specific examples of these esters include dibutyl adipate, di(2-ethylhexyl) sebacate, di-n-hexyl fumarate, dioctyl sebacate, diisoctyl azelate, dioctyl phthalate, didecyl phthalate, dieicosyl sebacate, the 2-ethylhexyl diester of linoleic acid dimer, and the complex ester formed by reacting one mole of sebacic acid with two moles of tetraethylene glycol and two moles of 2-ethyl-hexanoic acid.

The ratio of oil to water in the final formulated metalworking fluid (if oil is present) may vary from about 1:5 to about 1:200. Other oil-in-water emulsifier may be used supplementally (for particular performance characteristics) in preparing the emulsions of the present invention. Emulsifiers may be single materials or may be mixtures of surfactants. General emulsifiers include alkali metal sulfonates and carboxylates, salts derived from the reaction product of carboxylic acylating agents with amines and hydroxylamines, polyols, polyether glycols, polyethers, and polyesters and the like. The Kirk-Othmer Encyclopedia of Chemical Technology (3rd. Edition V. 8 pp. 900-930) provides a good discussion of emulsions and provides a list of emulsifiers useful in preparation of oil-in-water emulsions.

A typical metal working fluid would include other components such as anti-foam agents, metal deactivators, corrosion inhibitors, antimicrobial, extreme pressure, antiwear, antifriction, and antirust agents. Typical anti-friction agents include overbased sulfonates, sulfurized olefins, chlorinated paraffins and olefins, sulfurized ester olefins, amine terminated polyglycols, and sodium dioctyl phosphate salts. Useful anti-foam agents include: alkyl polymethacrylates, and polymethylsiloxanes. Metal deactivators include materials such as tolyltriazoles. Corrosion inhibitors include carboxylic/boric acid diamine salts, carboxylic acid amine salts, alkanol amines, alkanol amine borates and the like.

As used herein, the term “hydrocarbyl substituent” or “hydrocarbyl group” is used in its ordinary sense, which is well-known to those skilled in the art. Specifically, it refers to a group having a carbon atom directly attached to the remainder of the molecule and having predominantly hydrocarbon character.

Examples of hydrocarbyl groups include: (1) hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form a ring);

(2) substituted hydrocarbon substituents, that is, substituents containing non-hydrocarbon groups which, in the context of this invention, do not alter the predominantly hydrocarbon substituent (e.g., halo (especially chloro and fluoro), hydroxy, alkoxy, mercapto, alkylmercapto, nitro, nitroso, and sulfoxy);

(3) hetero substituents, that is, substituents which, while having a predominantly hydrocarbon character, in the context of this invention, contain other than carbon in a ring or chain otherwise composed of carbon atoms. Heteroatoms include sulfur, oxygen, nitrogen, and encompass substituents as pyridyl, furyl, thienyl and imidazolyl. In general, no more than two, preferably no more than one, non-hydrocarbon substituent will be present for every ten carbon atoms in the hydrocarbyl group; typically, there will be no non-hydrocarbon substituents in the hydrocarbyl group.

It is known that some of the materials described above may interact in the final formulation, so that the components of the final formulation may be different from those that are initially added. For instance, metal ions can migrate to other acidic sites of other molecules. The products formed thereby, including the products formed upon employing the composition of the present invention in its intended use, may not susceptible of easy description. Nevertheless, all such modifications and reaction products are included within the scope of the present invention; the present invention encompasses the composition prepared by admixing the components described above.

Each of the documents referred to above is incorporated herein by reference. Except in the Examples, or where otherwise explicitly indicated, all numerical quantities in this description specifying amounts of materials, reaction conditions, molecular weights, number of carbon atoms, and the like, are to be understood as modified by the word “about.” Unless otherwise indicated, each chemical or composition referred to herein should be interpreted as being a commercial grade material which may contain the isomers, by-products, derivatives, and other such materials which are normally understood to be present in the commercial grade. However, the amount of each chemical component is presented exclusive of any solvent or diluent oil which may be customarily present in the commercial material, unless otherwise indicated. It is to be understood that the upper and lower amount, range, and ratio limits set forth herein may be independently combined. As used herein, the expression “consisting essentially of” permits the inclusion of substances which do not materially affect the basic and novel characteristics of the composition under consideration.

What is claimed is:

1. An emulsifier for forming an oil in water emulsion comprising
a) a reaction product of maleic anhydride and a triglyceride oil from a plant or land animal
b) further reacted with water, Group IA and IIA metals, ammonium hydroxide, various amines, alkanolamines, polyols, alkoxylated alkanolamines, poly(alkylene oxide)s, or polyamines or mixtures thereof to form an emulsifier.

2. An emulsifier according to claim 1, wherein there is an average of 0.1 to 2 mole of succinate groups per mole of triglyceride oil.

3. An emulsifier according to claim 2, wherein said reaction product is reacted with an alkanolamine.

4. An emulsifier according to claim 2, wherein said reaction product is reacted with a polyamine.

5. An emulsifier according to claim 2, wherein said reaction product is reacted with ammonium hydroxide.

6. An emulsifier according to claim 2, wherein said reaction product is reacted with triethanolamine.

7. A metalworking fluid comprising;
a) a reaction product of maleic anhydride and a triglyceride oil from a plant or land animal, optionally further reacted with water, Group IA and IIA metals, ammonium hydroxide, various amines, alkanolamines, and polyamines to form an emulsifier,
b) a major amount of water, and
c) optionally an oil selected from the group consisting of triglyceride oils, hydrocarbon oils (aliphatic, aromatic, petroleum distillates, poly(alpha olefins), Fischer Tropsch oils, etc), and ester oils other than triglyceride oils.

8. A metalworking fluid according to claim 7, wherein said reaction product is present in an amount from about 0.5 to about 10 weight percent based on the weight of said fluid.

9. A metalworking fluid according to claim 8, wherein said reaction product has from about 0.1 to about 2 mole of succinate groups per mole of triglyceride oil.

10. A metalworking fluid according to claim 8, further comprising at least one of a corrosion inhibiting agent and/or a antiwear agent.

11. A metalworking fluid according to claim 10, further comprising an extreme pressure agent.

12. A metalworking fluid according to claim 10, further comprising a biocide.

13. A metalworking fluid according to claim 7, wherein said optional oil is present from about 1 to about 50 weight percent based on the weight of said metalworking fluid.

14. A metalworking fluid according to claim 8, wherein said reaction product of maleic anhydride and triglyceride oil forms a succinated triglyceride oil which is then is reacted with an alkanolamine to form some a) ester linkages between the succinic acid of said succinated triglyceride and the alcohol of the alkanolamine and b) some salts of the succinic acid of said succinated triglyceride and the nitrogen atom of the alkanolamine.

15. A metalworking fluid according to claim 14, wherein said alkanolamine comprises triethanolamine.

16. A metalworking fluid according to claim 14, wherein said alkanolamine comprises an ethoxylated triethanolamine.

17. A metalworking fluid according to claim 14, wherein said alkanolamine comprises an alkanolamine with two alcohol substituents and one alkyl substituent or two alkyl substituents and one alcohol substituent.

18. A metalworking fluid according to claim 7, wherein said reaction product of maleic anhydride and a triglyceride oil comprises both the reaction product of a first triglyceride oil and maleic anhydride and the reaction product of a second different triglyceride oil and maleic anhydride.

19. A metalworking fluid according to claim 7, wherein said component c) comprises a triglyceride oil of a plant or land animal that hasn't been reacted with maleic anhydride.

20. A metalworking fluid according to claim 7, where said reaction product of maleic anhydride and a triglyceride oil from a plant or land animal is further reacted with an ethoxylated or propoxylated alkanolamine forming a half-ester salt.
